(19) United States
(12) Patent Application Publication
(10) Pub. No.: US 2019/0008705 A1
(43) Pub. Date: Jan. 10, 2019

(54) PRESSURE-SENSITIVE ADHESIVE FASTENER, FASTENER, ABSORBENT ARTICLE, ADHESIVE MATERIAL ADHERING TO CLOTHING, AND ADHESIVE MATERIAL ADHERING TO FABRIC

(71) Applicant: 3M INNOVATIVE PROPERTIES COMPANY, St Paul, MN (US)

(72) Inventor: Shin Katoh, Tokyo (JP)

(73) Assignee: 3M INNOVATIVE PROPERTIES COMPANY, St. Paul, MN (US)

(21) Appl. No.: 16/066,477

(22) PCT Filed: Dec. 28, 2016
(86) PCT No.: PCT/US2016/068179

§ 371 Date: Jun. 27, 2018

(30) Foreign Application Priority Data
Dec. 28, 2015 (JP) 2015-256908

(51) Int. Cl. A61F 13/56; A44B 18/00; A61F 13/58

(52) U.S. Cl. CPC A61F 13/5611; A61F 13/581; A44B 18/0073; A44B 18/0049

(57) ABSTRACT

A pressure-sensitive adhesive fastener (100) is overlapped with a target object (150) on one surface (110a) side provided with a pressure-sensitive adhesive layer (121) and protruding members (130) to be coupled with the target object (150). With the pressure-sensitive adhesive fastener (100) coupled with the target object (150), the pressure-sensitive adhesive layer (121) is bonded to the target object (150), and part of column sections (131) and insertion sections (132) of the protruding members (130) enter gaps in the target object (150). This entrance creates such a mode that the protruding members (130) are stuck into the target object (150), resulting in a prevention of a shift in position between the pressure-sensitive adhesive fastener (100) and the target object (150).

### TECHNICAL FIELD

An aspect of the present invention relates to pressure-sensitive adhesive fasteners, fasteners, absorbent articles, adhesive materials adhering to clothing, and adhesive materials adhering to fabric.

### BACKGROUND ART

Conventionally, pressure-sensitive adhesives have been widely used to secure articles to clothing or the like. For example, absorbent articles, such as sanitary napkins and slight incontinence pads, are secured to clothing, such as underwear, via pressure-sensitive adhesives. Patent Document 1 discloses a pressure-sensitive adhesive fastener intended to attach an article to clothing in a peelable manner. This pressure-sensitive adhesive fastener includes protruding members that are pointed so as to have tapered tips and protrude from a pressure-sensitive adhesive layer.

### PATENT LITERATURE

Patent Document 1: Japanese Unexamined Patent Application Publication No. H2-298569A

### SUMMARY OF INVENTION

When an article (such as a sanitary napkin) is secured to clothing or fabric, it is expected that the article is prevented from shifting in position, the clothing or fabric is prevented from being damaged in peeling, the pressure-sensitive adhesive is prevented from remaining on the clothing or fabric after peeling (a residual adhesive is reduced), and the article readily adheres to again. Concerning these expectations, conventional securing only with a pressure-sensitive adhesive provides high adhesive strength to prevent a shift in position and thus affects damage to clothing or fabric, a residual adhesive, re-adhesion, and the like. Securing with the pressure-sensitive adhesive fastener disclosed in Patent Document 1, for example, causes a shift in position during wearing in some cases. A pressure-sensitive adhesive fastener and a fastener are thus required that prevent a shift in position, reduce damage to a target object in peeling, reduce a residual pressure-sensitive adhesive on the target object after peeling, and can readily adhere again.

### Solution to Problem

A pressure-sensitive adhesive fastener according to an aspect of the present invention, being overlapped with a target object to be coupled with the target object, includes: a sheet base material including a pair of facing surfaces; a pressure-sensitive adhesive layer being formed on at least one of the surfaces of the sheet base material; and a plurality of protruding members protruding from the at least one surface of the sheet base material in a first direction and being arranged so as to be separated from each other in a second direction being an expanding direction of the one surface. The protruding members each include: a column section extending from the sheet base material in the first direction and protruding from a front surface of the pressure-sensitive adhesive layer; and an insertion section protruding from a tip portion of the column section in the second direction and being inserted into the target object.

The pressure-sensitive adhesive fastener according to the aspect is overlapped with the target object on one surface side provided with the pressure-sensitive adhesive layer and the protruding members to be coupled with the target object. With the pressure-sensitive adhesive fastener coupled with the target object, the pressure-sensitive adhesive layer is bonded to the target object, and part of the column sections and the insertion sections of the protruding members enter gaps in the target object (clothing or fabric, for example, stitches, threads, or fibers of a cotton cloth of underwear). This entrance creates such a mode that the protruding members are stuck into the target object, resulting in a prevention of a shift in position between the pressure-sensitive adhesive fastener and the target object. The prevention of a shift in position reduces the necessity of using a strong adhesive, and/or the protruding members interfering with the target object prevent excess adhesion of the pressure-sensitive adhesive layer to the target object, resulting in a reduction in a residual pressure-sensitive adhesive on the target object after peeling. The reduction in the necessity of using a strong adhesive and/or the protruding parts having such a shape as not to be entangled with the target object reduces damage to the target object in peeling. The reduction in the necessity of using a strong adhesive and/or a reduction in an adhering area allows the fastener peeled off after once having adhered to the target object to readily adhere again.

According to an aspect, the insertion section may include an edge on an outer peripheral side in the second direction, a first lateral side being disposed on a distal end side of the column section with respect to the edge, and a second lateral side being disposed on a proximal end side of the column section with respect to the edge. A tip surface of the column section and the first lateral side of the insertion section may form a continuous surface together. This configuration enables the insertion sections to be positioned close to the tip surfaces of the column sections, resulting in a ready insertion of the insertion sections into gaps in the target object when the tip surfaces of the column sections abut on the target object. Consequently, the protruding members are readily stuck into the target object.

According to an aspect, the tip surface of the column section and the first lateral side of the insertion section may form a same plane. This configuration enables the insertion sections to be in the same position as the tip surfaces of the column sections, resulting in a ready insertion of the insertion sections into gaps in the target object when the tip surfaces of the column sections abut on the target object. Consequently, the protruding members are readily stuck into the target object.

According to an aspect, the insertion section may include an edge on an outer peripheral side in the second direction, a first lateral side being disposed on a distal end side of the column section with respect to the edge, and a second lateral side being disposed on a proximal end side of the column section with respect to the edge. The edge may be composed of a corner between the first lateral side and the second lateral side. The corner may be at an acute angle in a cross section cut in the first direction. This configuration enables each of the insertion sections to have a corner at an acute angle, resulting in a ready insertion into gaps in the target object. Consequently, the protruding members are readily stuck into the target object.

According to an aspect, the second lateral side is formed into an inward recess in the cross section cut in the first direction.

According to an aspect, the insertion section may include an edge on an outer peripheral side in the second direction, a first lateral side being disposed on a distal end side of the column section with respect to the edge, and a second lateral side being disposed on a proximal end side of the column section with respect to the edge. At least one of the first lateral side and the second lateral side may be formed into an outward protrusion in a cross section cut in the first direction. According to an aspect, the pressure-sensitive adhesive fastener may have a dynamic shear strength of 7 N/6.45 cm² or greater on a cotton cloth. This configuration can prevent a shift in position.

A pressure-sensitive adhesive fastener according to an aspect of the present invention, being overlapped with a target object to be coupled with the target object, includes: a sheet base material including a pair of facing surfaces; a pressure-sensitive adhesive layer being formed on at least one of the surfaces of the sheet base material; and a plurality of protruding members protruding from the at least one surface of the sheet base material in a first direction and being arranged so as to be separated from each other in a second direction being an expanding direction of the one surface. The protruding members each include: a wide portion being widened in the second direction on a distal end side; and a narrow portion being narrower than the wide portion in the second direction on a proximal end side. The wide portion has a width of 250 μm or less. The narrow portion has a width of 95% or less of the width of the wide portion. A difference between an average height of the protruding members (from the sheet base material) and an average thickness of the pressure-sensitive adhesive (from the sheet base material) is 250 μm or less.

The pressure-sensitive adhesive fastener according to the aspect is overlapped with the target object, such as clothing, on one surface side provided with the pressure-sensitive adhesive layer and the protruding members to be coupled with the target object. With the pressure-sensitive adhesive fastener coupled with the target object, the pressure-sensitive adhesive layer is bonded to the target object, and the wide portions, having the above-described configuration, of the protruding members enter gaps in the target object. This entrance creates such a mode that the protruding members are stuck into the target object, resulting in a prevention of a shift in position between the pressure-sensitive adhesive fastener and the target object. The prevention of a shift in position reduces the necessity of using a strong adhesive, and/or the protruding members prevent excess adhesion of the pressure-sensitive adhesive layer to the target object, resulting in a reduction in a residual pressure-sensitive adhesive on the target object after peeling. The reduction in the necessity of using a strong adhesive and/or the protruding parts having such a shape as not to be entangled with the target object reduces damage to the target object in peeling. The reduction in the necessity of using a strong adhesive and/or a reduction in an adhering area allows the fastener peeled off after once having adhered to the target object to readily adhere again.

An absorbent article according to an aspect of the present invention includes the pressure-sensitive adhesive fastener.

An adhesive material adhering to clothing according to an aspect of the present invention includes the pressure-sensitive adhesive fastener.

An adhesive material adhering to fabric according to an aspect of the present invention includes the pressure-sensitive adhesive fastener.

A fastener according to an aspect of the present invention, being overlapped with a target object to be coupled with the target object, includes: a sheet base material including a pair of facing surfaces; and a plurality of protruding members protruding from at least one of the surfaces of the sheet base material in a first direction and being arranged so as to be separated from each other in a second direction being an expanding direction of the one surface. The protruding members each include: a column section extending from the sheet base material in the first direction; and an insertion section protruding from a tip portion of the column section in the second direction and being inserted into the target object.

According to an aspect, the insertion section may include an edge on an outer peripheral side in the second direction, a first lateral side being disposed on a distal end side of the column section with respect to the edge, and a second lateral side being disposed on a proximal end side of the column section with respect to the edge. The edge may be composed of a corner between the first lateral side and the second lateral side. The corner may be at an acute angle in a cross section cut in the first direction.

The fastener according to these aspects can achieve the same action and effect as those of the above-described pressure-sensitive adhesive fasteners.

An absorbent article according to an aspect of the present invention includes the fastener.

An adhesive material adhering to clothing according to an aspect of the present invention includes the fastener.

An adhesive material adhering to fabric according to an aspect of the present invention includes the fastener.

### Advantageous Effects of Invention

The present invention provides a pressure-sensitive adhesive fastener that prevents a shift in position, reduces damage to a target object in peeling, reduces a residual pressure-sensitive adhesive on the target object after peeling, and allows an article to readily adhere again.

### BRIEF DESCRIPTION OF DRAWINGS

FIG. 1 illustrates a sanitary napkin including pressure-sensitive adhesive fasteners according to an embodiment.

FIG. 2 is a perspective view illustrating part of a pressure-sensitive adhesive fastener according to an embodiment.

FIG. 3 is a cross-sectional view illustrating part of a pressure-sensitive adhesive fastener according to an embodiment.

FIG. 4A is a perspective view illustrating the shape of a protruding member, and FIG. 4B is a cross-sectional view taken along the line IV-IV.

FIGS. 5A and 5B illustrate modified examples of a protruding member of a pressure-sensitive adhesive fastener according to an embodiment.

FIGS. 6A and 6B illustrate modified examples of a protruding member of a pressure-sensitive adhesive fastener according to an embodiment.

FIGS. 7A and 7B illustrate modified examples of a protruding member of a pressure-sensitive adhesive fastener according to an embodiment.

FIG. 8 shows results of an experiment to evaluate dynamic shear strength.

FIG. 9 shows results of an experiment to evaluate static shear strength.

### DESCRIPTION OF EMBODIMENTS

Various embodiments will now be described in detail with reference to the drawings. The same or equivalent components have the same reference characters in the drawings.

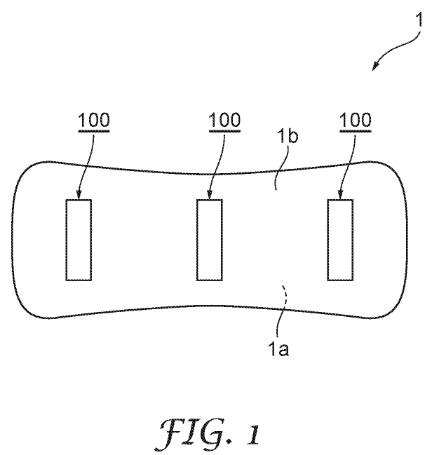

FIG. 1 illustrates a sanitary napkin including pressure-sensitive adhesive fasteners according to an aspect of the present invention. The sanitary napkin 1 includes a front surface 1a that faces a skin and has liquid permeability, a back surface 1b that faces clothing and has liquid impermeability, and pressure-sensitive adhesive fasteners 100 that are disposed on the back surface 1b. In the example illustrated in FIG. 1, the pressure-sensitive adhesive fasteners 100 are disposed in three regions on the back surface 1b while being separated from each other. The dimension of each of the regions provided with the pressure-sensitive adhesive fasteners 100 is approximately 1.5 cm×5.7 cm. The pressure-sensitive adhesive fastener 100 effectively prevents a shift in position, which will be described later, and accordingly the necessity of disposing the pressure-sensitive adhesive fastener 100 in a large area of the back surface 1b is reduced. The number of the regions provided with the pressure-sensitive adhesive fasteners 100, and the shape and dimension of each of the regions are not limited to those described above and may be designed in a desired manner. An article to which the pressure-sensitive adhesive fastener 100 is applied is not limited to a sanitary napkin, and the pressure-sensitive adhesive fastener 100 may be applied to, for example, a slight incontinence pad, a pocket warmer (an article used by adhering to clothing), or a tag (an article used by adhering to a curtain or the like). Articles including sanitary napkins and slight incontinence pads are referred to as absorbent articles. Articles including pocket warmers and the like used by adhering to clothing as well as the absorbent articles are referred to as adhesive materials adhering to clothing. Articles including tags and the like used by adhering to curtains or the like as well as the adhesive materials adhering to clothing are referred to as adhesive materials adhering to fabric.

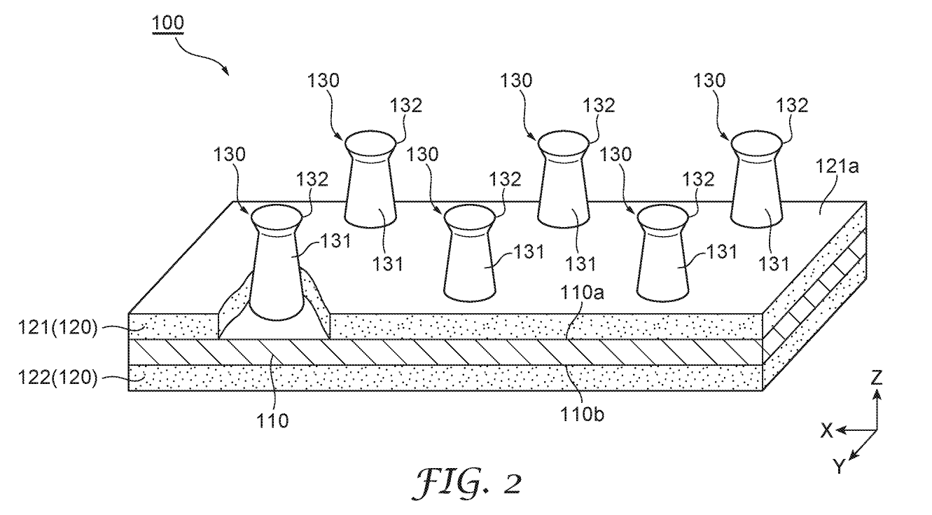

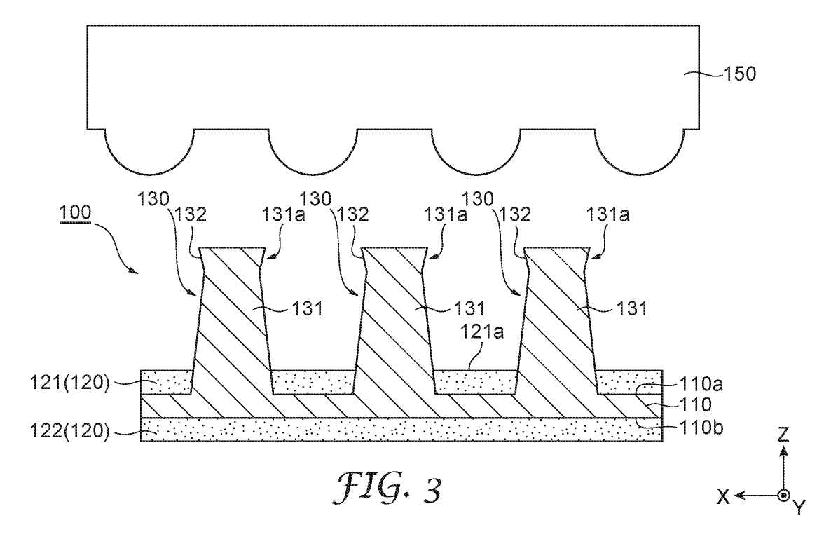

A configuration of the pressure-sensitive adhesive fastener 100 will now be described with reference to FIGS. 2 and 3. FIG. 2 is a perspective view illustrating part of the pressure-sensitive adhesive fastener 100 according to an embodiment. FIG. 3 is a cross-sectional view illustrating part of the pressure-sensitive adhesive fastener 100 according to an embodiment. For convenience of the description, XYZ orthogonal coordinate systems are also indicated in FIGS. 2 and 3.

The pressure-sensitive adhesive fastener 100 is overlapped with a target object 150, such as clothing, to be coupled with the target object 150. This coupling allows the pressure-sensitive adhesive fastener 100 to secure the sanitary napkin 1 to the target object 150, such as clothing. If the target object 150 is a cotton cloth, the pressure-sensitive adhesive fastener 100 may have a dynamic shear strength of 7 N/6.45 cm² or greater, 8 N/6.45 cm² or greater, or 9 N/6.45 cm² or greater on the cotton cloth. As illustrated in FIGS. 2 and 3, the pressure-sensitive adhesive fastener 100 includes a sheet base material 110, pressure-sensitive adhesive layers 120, and a plurality of protruding members 130. In the present embodiment, the protruding direction of the protruding members 130 is a Z-axis direction (first direction). A direction orthogonal to the Z-axis direction is an X-axis direction, and the direction orthogonal to the Z-axis direction and the X-axis direction is a Y-axis direction.

The sheet base material 110 is substantially sheet shaped and includes a first surface 110a and a second surface 110b that face each other in the Z-axis direction. The sheet base material 110 expands in an XY-plane direction (second direction). That is, the first surface 110a and the second surface 110b expand in the XY-plane direction. The protruding members 130 are formed on the first surface 110a between these surfaces. The sheet base material 110 is made from, for example, a resin material, such as poly(ethylene terephthalate), polyamide such as nylon, poly(styrene-acrylonitrile), poly(acrylonitrile-butadiene-styrene), polyolefin such as polypropylene, plasticized vinyl chloride, and polyester. The sheet base material 110 has a thickness of approximately 20 μm to 500 μm or 20 μm to 150 μm.

The pressure-sensitive adhesive layers 120 are formed on both the first surface 110a and the second surface 110b of the sheet base material 110. Here, the pressure-sensitive adhesive layer 120 formed on the first surface 110a is referred to as a pressure-sensitive adhesive layer 121, and the pressure-sensitive adhesive layer 120 formed on the second surface 110b is referred to as a pressure-sensitive adhesive layer 122. The pressure-sensitive adhesive layer 121 is provided so as to fill gaps between the protruding members 130. The pressure-sensitive adhesive layers 121, 122 are formed so as to cover substantially the entire surfaces 110a, 110b (except the positions of the protruding members 130). The pressure-sensitive adhesives of the pressure-sensitive adhesive layer 121 and the pressure-sensitive adhesive layer 122 may be of mutually different types. The pressure-sensitive adhesive layer 122 may not be provided in some cases. The pressure-sensitive adhesives may also be attached to lateral sides and top surfaces of the protruding members 130 depending on production processes and storage conditions. Materials of the pressure-sensitive adhesive layers 121, 122 include, for example, natural rubber/resin, synthetic rubber/resin, and acrylate copolymers. The pressure-sensitive adhesive layers 121, 122 each have a thickness of approximately 15 μm to 100 μm or 15 μm to 50 μm.

The protruding members 130 protrude from the first surface 110a of the sheet base material 110 in the Z-axis direction (first direction). The protruding members 130 are arranged so as to be separated from each other in the XY-plane direction (second direction) in which the first surface 110a expands. The arrangement is not limited to an orthogonal lattice in the X and Y directions and may be in various patterns, such as an oblique lattice in the X and Y directions, a staggered pattern, and a random pattern. Materials of the protruding members 130 include, for example, poly(ethylene terephthalate), polyamide such as nylon, poly(styrene-acrylonitrile), poly(acrylonitrile-butadiene-styrene), polyolefin such as polypropylene, plasticized vinyl chloride, and polyester. The protruding members 130 may be made from the same material as that of the sheet base material 110 and formed integrally with the sheet base material 110. Alternatively, the protruding members 130 may be made from a different material from that of the sheet base material 110 and formed as separate members. The density of the arranged protruding members 130 is, for example, approximately 500 to 5000 pieces or 1600 to 3500 pieces per square inch.

The protruding members 130 protrude far on the Z-axial positive side with respect to a front surface 121a, on the Z-axial positive side, of the pressure-sensitive adhesive layer 121. The average height of the protruding members 130 may be 500 μm or less. Alternatively, the average height may be 300 μm or less, 150 μm or less, or 100 μm or less. The average height of the protruding members 130 may be 20 μm or greater. Alternatively, the average height may be 40 μm or greater. The difference between the average height of the protruding members 130 (measured from the sheet base material 110) and the average thickness of the pressure-sensitive adhesive layer 121 (measured from the sheet base material 110) (in other words, the distance from the top surfaces, on the Z-axial positive side, of the protruding members 130 to the front surface 121a, on the Z-axial positive side, of the pressure-sensitive adhesive layer 121 in the Z-axis direction) may be 250 μm or less. Alternatively, the difference may be 200 μm or less, 150 μm or less, 100 μm or less, 50 μm or less, or 30 μm or less. The difference between the average height of the protruding members 130 and the average thickness of the pressure-sensitive adhesive layer 121 may be 10 μm or greater. Alternatively, the difference may be 20 μm or greater. This difference is finally determined within the spirit of the present invention depending on various factors, such as the dimension and density of the protruding members 130, the material and thickness of the pressure-sensitive adhesive, and the type of the target object. The height of all of the protruding members 130 in the pressure-sensitive adhesive fastener 100 may be the same (with variations within a production error allowed) or may be different between one region and the other region.

The protruding members 130 each include a column section 131 and an insertion section 132. The column section 131 extends from the sheet base material 110 in the Z-axis direction and protrudes from the front surface 121a of the pressure-sensitive adhesive layer 121. The insertion section 132 protrudes from a tip portion 131a of the column section 131 in the XY-plane direction and is inserted into the target object 150. The shapes of the column sections 131 and the insertion sections 132 of all of the protruding members 130 in the pressure-sensitive adhesive fastener 100 may be the same (with variations within a production error allowed) or may be different between one region and the other region.

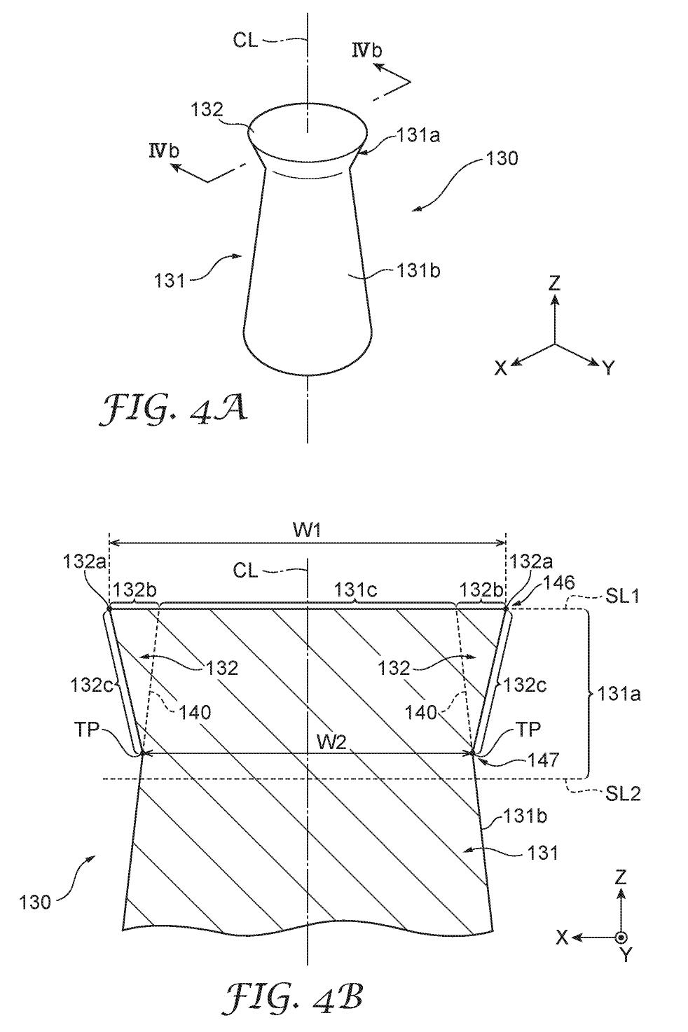

An aspect of the shape of the protruding members 130 will now be described in detail with reference to FIG. 4. FIG. 4A is a perspective view illustrating the protruding member 130, and FIG. 4B is a cross-sectional view taken along the line IV-IV in FIG. 4A. As illustrated in FIGS. 4A and 4B, the protruding member 130 has an axially symmetric shape around the central axis CL extending in the Z-axis direction.

As illustrated in FIG. 4B, the column section 131 according to the present embodiment has a truncated cone shape extending straight with its lateral side 131b tilting with respect to the central axis CL. However, the column section 131 may have any shape extending in the Z-axis direction. For example, the column section 131 may have a cylindrical shape extending in the Z-axis direction. Alternatively, the column section 131 may have such a shape that its lateral side 131b curves or changes in a stepwise manner in the Z-axis direction. Details of modified examples will be described later.

The column section 131 has a tip surface 131c being an end surface on the Z-axial positive side. The tip surface 131c according to the present embodiment is a plane expanding parallel to the XY-plane direction. The tip surface 131c may have any shape that does not affect securing performance of the protruding member 130 to the target object 150. For example, the tip surface 131c may have such a shape as to tilt or curve toward the Z-axial positive side (or negative side) on the central axis CL side. Alternatively, one region may tilt or curve, and the other may be planar. Details of modified examples will be described later.

The insertion section 132 includes an edge 132a that is on the outer peripheral side in the XY-plane direction, a first lateral side 132b that is disposed on the distal end side of the column section 131 with respect to the edge 132a, and a second lateral side 132c that is disposed on the proximal end side of the column section 131 with respect to the edge 132a.

The insertion section 132 may be formed integrally with the column section 131, such that a boundary (a boundary between the column section 131 and the insertion section 132) 140 is invisible. In the present embodiment, the boundary 140 is invisible and indicated by the broken line. Alternatively, the insertion section 132 may be formed separately from the column section 131 and then bonded to the column section 131, such that the boundary 140 is visible.

The lateral side of the protruding member 130 is tapered from the proximal end side toward the Z-axial positive side and widened from a point TP toward the Z-axial positive side. In the lateral side of the protruding member 130, the portion on the proximal end side with respect to the point TP corresponds to the lateral side 131b of the column section 131, and the portion on the distal end side with respect to the point TP corresponds to the second lateral side 132c of the insertion section 132. In the example in FIG. 4B, the tilted straight line extending from the lateral side 131b of the column section 131 defines the boundary 140. Accordingly, the portion on the outer peripheral side with respect to the boundary 140 corresponds to the insertion section 132, and the portion on the inner peripheral side with respect to the boundary 140 corresponds to the column section 131. On the tip surface of the protruding member 130, the portion on the outer peripheral side with respect to the boundary 140 corresponds to the first lateral side 132b of the insertion section 132, and the portion on the inner peripheral side with respect to the boundary 140 corresponds to the tip surface 131c of the column section 131. As described above, the boundary 140 may have the same shape as that of the column section 131 (here, the shape extending straight so as to be tapered). However, the boundary 140 may have any shape appropriately modified without departing from the spirit of the present invention. For example, the boundary 140 may be a straight line extending parallel to the Z axis from the point TP. In this case, the insertion section 132 provides the same function.

As illustrated in FIG. 4B, the first lateral side 132b is a plane expanding from the tip surface 131c of the column section 131 to the outer peripheral side. This configuration enables the first lateral side 132b and the tip surface 131c (also referred to as a top surface) of the column section 131 to form a continuous surface together. Here, the first lateral side 132b and the tip surface 131c of the column section 131 form the same plane (with variations within a production error allowed). The first lateral side 132b may have any shape and may be a tilting surface, a curving surface, or a plurality of tilting surfaces changing in a stepwise manner. Details of modified examples will be described later. The first lateral side 132b and the tip surface 131c of the column section 131 may not form a continuous surface. For example, a groove, a step, or the like may be formed between the first lateral side 132b and the tip surface 131c.

The second lateral side 132c expands straight from the point TP at a certain tilt angle toward the Z-axial positive side. If the first lateral side 132b is a plane parallel to the XY-plane direction as in the present embodiment, the tilt angle of the second lateral side 132c (the angle to the first lateral side 132b) may be an acute angle, 0.1 to 89.9°, 1 to 80°, or 3 to 70°. The first lateral side 132b may have any shape and may be a curving surface or a plurality of tilting surface changing in a stepwise manner. Details of modified examples will be described later.

The edge 132a forms a peak on the outer peripheral side in the XY-plane direction, in the insertion section 132. In the present embodiment, the edge 132a is composed of a corner between the first lateral side 132b and the second lateral side 132c. The edge 132a is thus positioned on the outermost peripheral side in the insertion section 132. The corner composing the edge 132a is at an acute angle in a cross section cut in the Z-axis direction (cut along a plane extending in the Z-axis direction and including the central axis CL). The edge 132a may not be composed of a corner. The cross-sectional view illustrates the edge 132a as a point; however, the edge 132a may be illustrated as a straight line parallel to the central axis CL as long as the edge 132a is positioned on the outermost peripheral side. In this case, the straight line (parallel to the central axis CL) illustrating the edge 132a is preferably short such that the insertion section 132 can be inserted into the front surface of the target object. Details of modified examples will be described later.

A Z-axial position of the edge 132a will now be described. The edge 132a is preferably disposed as close as possible to the tip of the column section 131. This configuration allows a portion in the vicinity of the edge 132a in the insertion section 132 to be readily inserted into a gap of the target object 150 when the protruding member 130 abuts on the target object 150. If the edge 132a of the insertion section 132 is disposed remote from the tip of the column section 131 on the Z-axial negative side, the insertion section 132 is difficult to be inserted into a gap of the target object 150 or cannot be inserted thereinto. Thus, if a region within a predetermined range from the tip of the column section 131 is defined as the tip portion 131a, the edge 132a is preferably disposed in the region defined as the tip portion 131a in the Z-axis direction. For example, a straight line that passes through the tip of the column section 131 (a peak on the Z-axial positive side; the entire tip surface 131c corresponds to the peak in the present embodiment) and that is parallel to the XY-plane direction is represented by a reference line SL1. A reference line SL2 parallel to the XY-plane direction is defined in a position remote from the reference line SL1 toward the Z-axial negative side by a predetermined distance. The region between the reference line SL1 and the reference line SL2 is defined as the tip portion 131a. If the target object 150 is a cotton cloth, the tip portion 131a may have a Z-axial dimension of 100 μm or less, 75 μm or less, 50 μm or less, or 25 μm or less. The insertion section 132 may have any dimension on the Z-axial negative side as long as it does not affect the performance. The point TP being the edge, on the Z-axial negative side, of the insertion section 132 is positioned on the Z-axial positive side with respect to the reference line SL2 in the drawing but may be positioned on the reference line SL2 or on the Z-axial negative side with respect to the reference line SL2.

The protruding member 130 configured as described above includes a wide portion 146 that is widened in the XY-plane direction on the distal end side and a narrow portion 147 that is narrower than the wide portion 146 in the XY-plane direction on the proximal end side. In the present embodiment, the wide portion 146 is formed in the position where the edge 132a of the insertion section 132 is formed. The narrow portion 147 is formed between the insertion section 132 and the column section 131 (in the position of the point TP). The width W1 of the wide portion 146 may be 250 μm or less. Alternatively, the width W1 may be 200 μm or less, 150 μm or less, or 100 μm or less. The width W2 of the narrow portion 147 may be 95% or less of the width W1 of the wide portion 146. Alternatively, the width W2 may be 90% or less, or 85% or less. The width W2 of the narrow portion 147 may be 20% or greater of the width W1 of the wide portion 146. Alternatively, the width W2 may be 35% or greater, 50% or greater, 60% or greater, or 70% or greater.

A method of producing the pressure-sensitive adhesive fastener 100 will now be described. First, the sheet base material 110 and an original form of the protruding members 130 are integrally molded. The original form of the protruding members 130 (for example, a columnar object) is processed, for example, by pressurization to be molded into the protruding members 130. Alternatively, the sheet base material 110 and the protruding members 130 may be integrally molded in one process. Second, the pressure-sensitive adhesive layers 121, 122 are formed on the first surface 110a and the second surface 110b of the sheet base material 110 (See also Patent Document 1: Japanese Unexamined Patent Application Publication No. H2-298569A). In this case, the pressure-sensitive adhesive layers may be charged from above (the Z direction), from a side (the X and Y directions), or from any other direction, such as a slanting direction.

The action and effect of the pressure-sensitive adhesive fastener 100 according to the present embodiment will now be described.

The pressure-sensitive adhesive fastener 100 according to the present embodiment is overlapped with the target object 150 on one surface 110a side provided with the pressure-sensitive adhesive layer 121 and the protruding members 130 to be coupled with the target object 150. With the pressure-sensitive adhesive fastener 100 coupled with the target object 150, the pressure-sensitive adhesive layer 121 is bonded to the target object 150, and part of the column sections 131 and the insertion sections 132 of the protruding members 130 enter gaps in the target object 150. This entrance creates such a mode that the protruding members 130 are stuck into the target object 150, resulting in a prevention of a shift in position between the pressure-sensitive adhesive fastener 100 and the target object 150. The prevention of a shift in position reduces the necessity of using a strong adhesive, and/or the protruding members 130 interfering with the target object 150 prevent excess adhesion of the pressure-sensitive adhesive layer 121 to the target object 150, resulting in a reduction in a residual pressure-sensitive adhesive on the target object 150 after peeling. The reduction in the necessity of using a strong adhesive and/or the protruding parts having such a shape as not to be entangled with the target object reduces damage to the target object 150 in peeling. The reduction in the necessity of using a strong adhesive and/or a reduction in an adhering area allows the fastener peeled off after once having adhered to the target object 150 to readily adhere again.

In the pressure-sensitive adhesive fastener 100 according to the present embodiment, the insertion sections 132 each include the edge 132a that is on the outer peripheral side in the XY-plane direction, the first lateral side 132b that is disposed on the distal end side of the column section 131 with respect to the edge 132a, and the second lateral side 132c that is disposed on the proximal end side of the column section 131 with respect to the edge 132a. The tip surfaces 131c of the column sections 131 and the first lateral sides 132b of the insertion sections 132 each form a continuous surface together. This configuration enables the insertion sections 132 to be positioned close to the tip surfaces 131c of the column sections 131, resulting in a ready insertion of the insertion sections 132 into gaps in the target object 150 when the tip surfaces 131c of the column sections 131 abut on the target object 150. Consequently, the protruding members 130 are readily stuck into the target object 150.

In the pressure-sensitive adhesive fastener 100 according to the present embodiment, the tip surfaces 131c of the column sections 131 and the first lateral sides 132b of the insertion sections 132 each forms the same plane. This configuration enables the insertion sections 132 to be in the same position as the tip surfaces 131c of the column sections 131, resulting in a ready insertion of the insertion sections 132 into gaps in the target object 150 when the tip surfaces 131c of the column sections 131 abut on the target object 150. Consequently, the protruding members 130 are readily stuck into the target object 150.

In the pressure-sensitive adhesive fastener 100 according to the present embodiment, the edges 132a are each composed of a corner between the first lateral side 132b and the second lateral side 132c. The corner is at an acute angle in the cross section cut in the Z-axis direction. This configuration enables each of the insertion sections 132 to have a corner at an acute angle, resulting in a ready insertion into gaps in the target object 150. Consequently, the protruding members 130 are readily stuck into the target object 150.

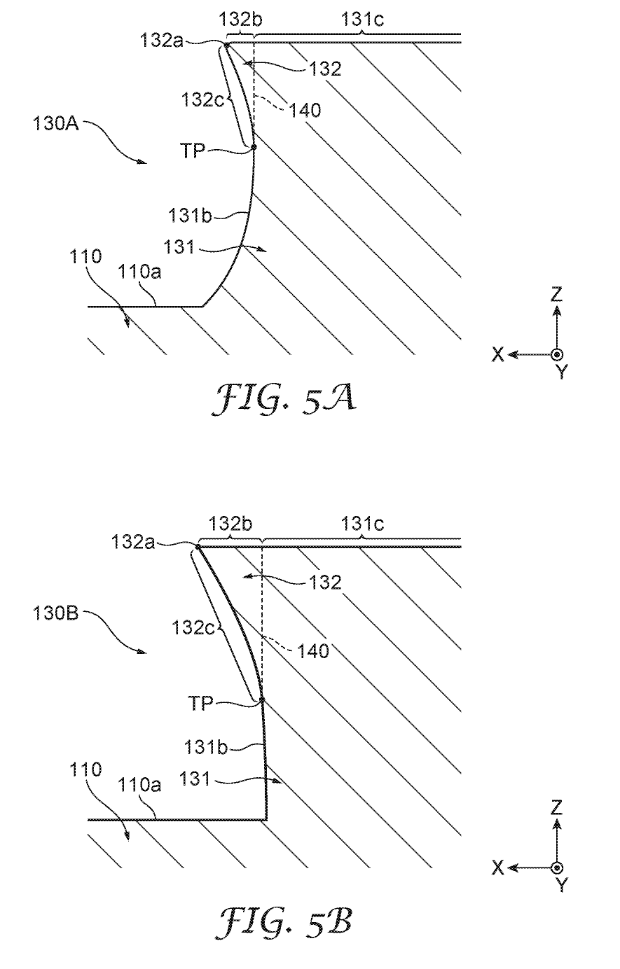

Shapes of protruding members of pressure-sensitive adhesive fasteners according to modified examples will now be described with reference to FIGS. 5A to 7B.

In a protruding member 130A illustrated in FIG. 5A, the lateral side 131b of the column section 131 and the second lateral side 132c of the insertion section 132 have shapes mainly different from those in the protruding member 130 illustrated in FIG. 4B. The configuration other than the above is the same; thus, its description is omitted.

The lateral side of the protruding member 130A illustrated in FIG. 5A is formed into an arc recessed inward in a cross section cut in the Z-axis direction. Here, a point TP represents a point of contact when a tangent to the arc at a predetermined point is parallel to the Z-axis direction. A straight line extending parallel to the Z-axis direction from the point TP thus defines the boundary 140. This configuration forms the lateral side 131b of the column section 131 and the second lateral side 132c into an inward recess in the cross section cut in the Z-axis direction. In the example illustrated in FIG. 5A, the corner composing the edge 132a is at an acute angle in the cross section cut in the Z-axis direction.

In a protruding member 130B illustrated in FIG. 5B, the lateral side 131b of the column section 131 and the second lateral side 132c of the insertion section 132 have shapes mainly different from those in the protruding member 130 illustrated in FIG. 4B. The configuration other than the above is the same; thus, its description is omitted.

In the lateral side of the protruding member 130B illustrated in FIG. 5B, the lateral side 131b of the column section 131 extends straight and the second lateral side 132c of the insertion section 132 is formed into an arc recessed inward in a cross section cut in the Z-axis direction. Here, a line extending from the lateral side 131b defines the boundary 140. The point where the lateral side is widened significantly is represented by a point TP. A straight line extending parallel to the Z-axis direction from the point TP thus defines the boundary 140. In the example illustrated in FIG. 5B, the corner composing the edge 132a is at an acute angle in the cross section cut in the Z-axis direction.

As described above, the shape of the lateral side 131b may be widened (toward the base) as illustrated in FIG. 5A or may not be widened as illustrated in FIG. 5B. This is also applied to the examples in FIGS. 4A, 4B, 6A, 6B, 7A, and 7B.

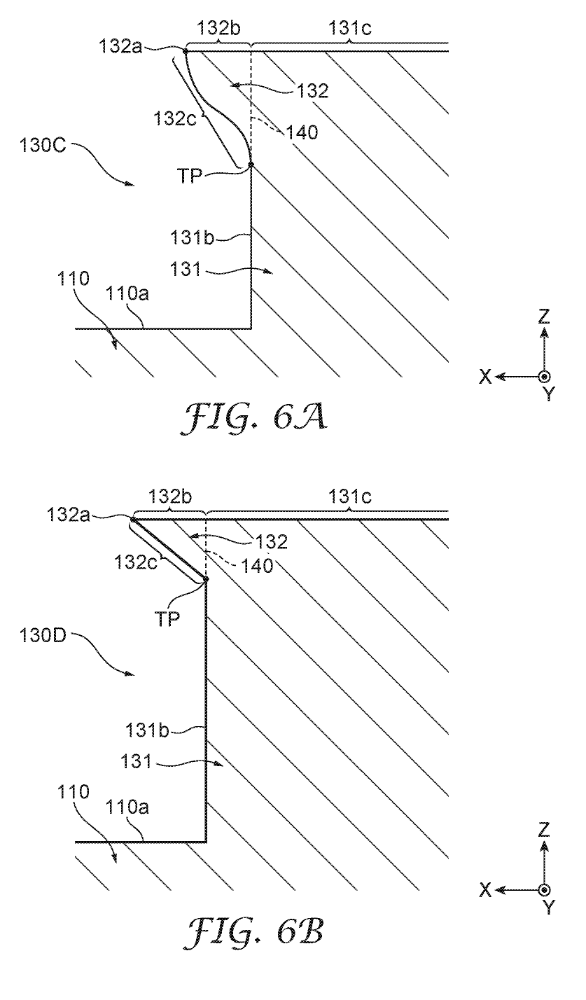

In a protruding member 130C illustrated in FIG. 6A, the lateral side 131b of the column section 131 and the second lateral side 132c of the insertion section 132 have shapes mainly different from those in the protruding member 130 illustrated in FIG. 4B. The configuration other than the above is the same; thus, its description is omitted.

In the lateral side of the protruding member 130C illustrated in FIG. 6A, the lateral side 131b of the column section 131 extends straight in the Z-axis direction in a cross section cut in the Z-axis direction, and the second lateral side 132c of the insertion section 132 is formed into an outward protrusion in the cross section cut in the Z-axis direction. In other words, the second lateral side 132c curves so as to protrude outward. Here, a line extending from the lateral side 131b defines the boundary 140. The point where the lateral side is widened significantly is represented by a point TP. A straight line extending parallel to the Z-axis direction from the point TP thus defines the boundary 140. In the example illustrated in FIG. 6A, the corner composing the edge 132a is at an acute angle in the cross section cut in the Z-axis direction.

In a protruding member 130D illustrated in FIG. 6B, the lateral side 131b of the column section 131 and the second lateral side 132c have shapes mainly different from those in the protruding member 130 illustrated in FIG. 4B. The configuration other than the above is the same; thus, its description is omitted.

In the lateral side of the protruding member 130D illustrated in FIG. 6B, the lateral side 131b of the column section 131 extends straight in the Z-axis direction in a cross section cut in the Z-axis direction. Here, a line extending from the lateral side 131b defines the boundary 140. The point where the diameter of the lateral side increases is represented by a point TP. A straight line extending parallel to the Z-axis direction from the point TP thus defines the boundary 140. In the example illustrated in FIG. 6B, the corner composing the edge 132a is at an acute angle in the cross section cut in the Z-axis direction.

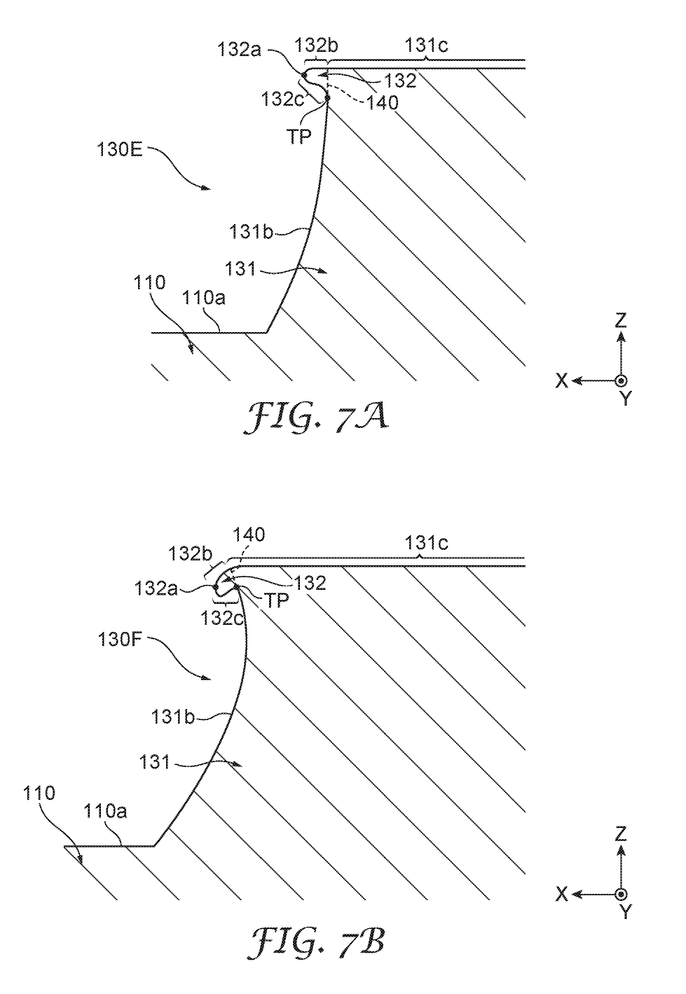

In a protruding member 130E illustrated in FIG. 7A, the lateral side 131b of the column section 131 and the first lateral side 132b and the second lateral side 132c of the insertion section 132 have shapes mainly different from those in the protruding member 130 illustrated in FIG. 4B. The configuration other than the above is the same; thus, its description is omitted.

In the lateral side of the protruding member 130E illustrated in FIG. 7A, the lateral side 131b of the column section 131 extends so as to be tapered toward the Z-axial positive side in a cross section cut in the Z-axis direction, and the second lateral side 132c of the insertion section 132 is formed into an outward protrusion in the cross section cut in the Z-axis direction. In other words, the second lateral side 132c curves so as to protrude outward. The first lateral side 132b of the insertion section 132 is formed into an outward protrusion in the cross section cut in the Z-axis direction. In other words, the first lateral side 132b curves so as to protrude outward. Here, a line extending from the lateral side 131b defines the boundary 140. The point where the lateral side is widened significantly is represented by a point TP. A straight line extending parallel to the Z-axis direction from the point TP thus defines the boundary 140.

In a protruding member 130F illustrated in FIG. 7B, the lateral side 131b of the column section 131 and the first lateral side 132b and the second lateral side 132c of the insertion section 132 have shapes mainly different from those in the protruding member 130 illustrated in FIG. 4B. The configuration other than the above is the same; thus, its description is omitted.

In the lateral side of the protruding member 130F illustrated in FIG. 7B, the lateral side 131b of the column section 131 extends so as to be tapered toward the Z-axial positive side on the proximal end side and so as to be widened toward the Z-axial positive side on the distal end side in a cross section cut in the Z-axis direction. The first lateral side 132b and the second lateral side 132c have a (small-width) portion extending from the distal end side to the proximal end side toward the outer peripheral side in the XY-plane direction. Here, a line extending from the lateral side 131b defines the boundary 140. The point where the lateral side is widened significantly is represented by a point TP. A straight line extending parallel to the Z-axis direction from the point TP thus defines the boundary 140.

### EXAMPLES

Experiments conducted to evaluate a pressure-sensitive adhesive fastener according to an aspect of the present invention will now be described.

### Experiment for Evaluation of Dynamic Shear Strength

This experiment is for measurement of dynamic shear strength of a commercial sanitary napkin including a general pressure-sensitive adhesive and a pressure-sensitive adhesive fastener according to the present invention that adhere to a target object. An effect of preventing a shift in position of the pressure-sensitive adhesive fastener is evaluated on the basis of dynamic shear strength in this experiment.

In this experiment, Working Example 7 and Working Example 8 shown in the fourth table of the prior art document, Japanese Unexamined Patent Application Publication No. H2-298569A, were used as Comparative Example 1 and Comparative Example 2, respectively. A 2.54×2.54 cm test piece was prepared as Comparative Example 3 by cutting a section, provided with the pressure-sensitive adhesive, of the commercial sanitary napkin. A test piece of the pressure-sensitive adhesive fastener having the same dimension as that of Comparative Example 3 was prepared as a working example. The pressure-sensitive adhesive fastener had the configuration illustrated in FIGS. 4A and 4B. The protruding members had a height of approximately 80 μm, a width W1 of approximately 200 μm, and a width W2 of approximately 160 μm. The corner composing the edge 132a was at an acute angle (in a cross section cut in the Z-axis direction). The pressure-sensitive adhesive layers were made from a synthetic rubber/resin and had a thickness of approximately 30 μm. That is, the difference between the height of the protruding members (measured from the sheet base material) and the thickness of the pressure-sensitive adhesive layer (measured from the sheet base material) was approximately 50 μm. An adherend fabric was prepared as the target object.

A 6.35×15.2 cm piece of the adherend fabric was attached to two ends of a steel panel. The 2.54 cm² test piece was disposed on the adherend fabric and pressure-bonded by running a 2.0 kg roll twice. The test piece was placed into a tensile tester and measured at a crosshead speed of 12.7 cm/min. Force required to cause shear adhesion failure between the test piece and the adherend fabric (dynamic shear strength) was measured in this way.

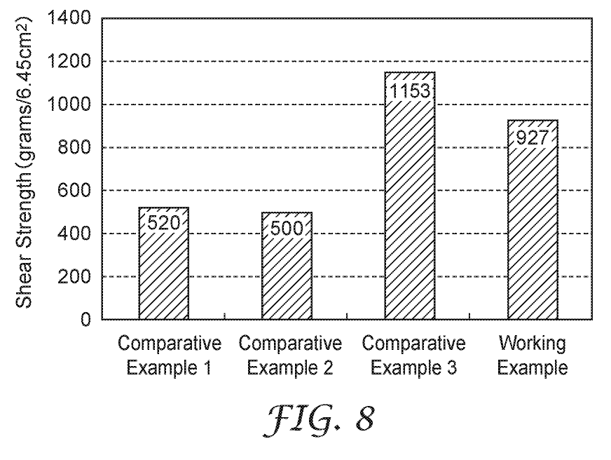

FIG. 8 shows the measurement results of dynamic shear strength. In FIG. 8, values of Working Example 7 and Working Example 8 shown in the fourth table of the prior art document, Japanese Unexamined Patent Application Publication No. H2-298569A, were cited as data of Comparative Example 1 and Comparative Example 2. The pressure-sensitive adhesive fastener 100 and the pressure-sensitive tape fastener disclosed in Japanese Unexamined Patent Application Publication No. H2-298569A can be compared with each other in this way. The dynamic shear strength of Comparative Example 1 and Comparative Example 2 were respectively 520 g/6.45 cm² (5.096 N/6.45 cm²) and 500 g/6.45 cm² (4.9 N/6.45 cm²). The dynamic shear strength of working examples was 927 g/6.45 cm² (9.085 N/6.45 cm²). The results confirmed that the pressure-sensitive adhesive fastener 100 prevented a shift in position more effectively than the pressure-sensitive tape fastener disclosed in the prior art document. The dynamic shear strength of the pressure-sensitive adhesive fastener 100 stood comparison with the dynamic shear strength of Comparative Example 3. The results confirmed that the pressure-sensitive adhesive fastener 100 had dynamic shear strength sufficient to prevent a shift in position. The conversion from g (gw) to N can be made with the relational expression: 1 gw=0.0098 N.

### Experiment for Evaluation of Static Shear Strength

This experiment is for measurement of static shear strength of a commercial sanitary napkin including a general pressure-sensitive adhesive and a pressure-sensitive adhesive fastener according to the present invention that adhere to a target object. An effect of preventing a shift in position of the pressure-sensitive adhesive fastener is evaluated on the basis of static shear strength in this experiment.

In this experiment, a 3×3 cm test piece was prepared as a comparative example by cutting a section, provided with the pressure-sensitive adhesive, of the commercial sanitary napkin as in Comparative Example 3 described above. A 3×3 cm test piece of the pressure-sensitive adhesive fastener (having the same configuration as that used as a working example in the evaluation of dynamic shear strength) was prepared as a working example. A 8×6 cm 100% cotton fabric was prepared as an adherend fabric.

To measure static shear strength, the adherend fabric was secured on a test board, and each of the test pieces was then placed statically on the adherend fabric. A weight to apply a load of normal force was placed via a 3×3 cm cork material on the test piece. The test piece was connected via a string to a cage where the weight to apply a tensile load was placed. After the test piece was set in this way, the test piece and the adherend fabric were pressure-bonded to each other with the cork material at a pressure of 3 g/cm² and then left for one minute as they were. Thereafter, the weight was placed in the cage to apply a 230 g load to the test piece. If the test piece did not move within one minute, a weight was added by 20 g at a time. A tensile load when the test piece moved within one minute was measured. Here, the term “move” does not indicate a slight shift but indicates that the test piece in comparative examples or working examples falls down.

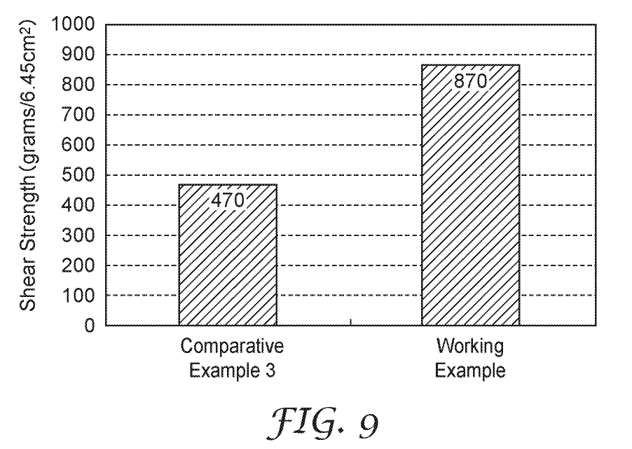

FIG. 9 shows the measurement results of static shear strength. The static shear strength of comparative examples was 470 g/9 cm² (4.606 N/9 cm²). The static shear strength of working examples was 870 g/9 cm² (8.526 N/9 cm²). The results confirmed that the pressure-sensitive adhesive fastener had static shear strength stronger than the general pressure-sensitive adhesive. The conversion from g (gw) to N can be made with the relational expression: 1 gw=0.0098 N.

### Experiment for Evaluation of Damage to Front Surface of Target Object

This experiment is for evaluation of damage to the front surface of a target object after peeling of a commercial sanitary napkin including a general pressure-sensitive adhesive and a sanitary napkin including pressure-sensitive adhesive fasteners according to the present invention.

In this experiment, the above-mentioned commercial sanitary napkin was prepared as a comparative example (sanitary napkin for comparative examples). A sanitary napkin including the pressure-sensitive adhesive fasteners according to working examples described above was prepared (sanitary napkin for working examples). The entire dimension of the sanitary napkin for working examples was approximately 22.5 cm in the longitudinal direction and approximately 9.2 cm in the lateral direction. The dimension of the pressure-sensitive adhesive for adhering the pressure-sensitive adhesive fasteners of working examples to the sanitary napkin was 1.5 cm in the longitudinal direction of the sanitary napkin and 5.7 cm in the lateral direction of the sanitary napkin. The pressure-sensitive adhesive fasteners were separated from each other by a predetermined distance on the sanitary napkin (See FIG. 1). The region of the pressure-sensitive adhesive on the back surface of the sanitary napkin for comparative examples was 15 cm in the longitudinal direction and 5.7 cm in the lateral direction. Two adherend fabrics were prepared as objects to which the sanitary napkins adhered. The adherend fabrics were 100% cotton fabrics having a dimension of 20 cm in the longitudinal direction and 7 cm in the lateral direction.

To evaluate damage to the target object, the sanitary napkin adhered to the adherend fabric, and the sanitary napkin and the adherend fabric were then disposed between two metal plates. Thereafter, a weight was placed on the upper metal plate so as to apply an average pressure of approximately 36 g/cm². In this state, the sanitary napkin and the adherend fabric were left at a temperature of 40° C. and a humidity of 80% for 24 hours. T-peel testing was then performed at room temperature. A tensile tester (TENSILON RTG-1225 produced by A&D Company, Limited) was used for the T-peel testing. The adherend fabric was secured to an upper chuck of the tensile tester, and the sanitary napkin was secured to a lower chuck. In this state, peeling was performed at a constant speed of 30 cm/min. After the T-peel testing, a peeled surface of the adherend fabric was observed with an optical microscope (VHX1000 produced by Keyence Corporation) with a magnification of 25 times. The height of fiber fluffing of the adherend fabric after the peeling was measured to evaluate damage to the adherend fabric.

The measurement results showed that the fluffing of the adherend fabric after peeling the sanitary napkin for comparative examples had an average height of 1.2 mm. The fluffing of the adherend fabric after peeling of the sanitary napkin for working examples had an average height of 0.8 mm. The results confirmed that the pressure-sensitive adhesive fastener caused less damage to the target object than the general pressure-sensitive adhesive.

### Experiment for Evaluation of Initial Adhesive Strength and Adhesive Strength After Adhesive Aging

This experiment is for measurement of initial adhesive strength (adhesive strength immediately after adhering) of a commercial sanitary napkin including a general pressure-sensitive adhesive and a sanitary napkin including a pressure-sensitive adhesive fastener according to the present invention. The experiment is also for measurement of adhesive strength after adhesive aging of each of the sanitary napkins to examine the relation with adhesive strength immediately after adhering.

In this experiment, two of each of the same sanitary napkins as those used in “Experiment for Evaluation of Damage to Front Surface of Target Object” were prepared. Four adherend fabrics were prepared as objects to which the sanitary napkins adhered. The adherend fabrics were 100% cotton fabrics having a dimension of 22.5 cm in the longitudinal direction and 7 cm in the lateral direction.

To measure initial adhesive strength, the adherend fabric was adhered such that one end of the adherend fabric in the longitudinal direction extends from one end of each of the sanitary napkins in the longitudinal direction by 3 cm. A pressure-bonding roller having a weight of 210 g and a width of 45 mm was then used to pressure-bond the sanitary napkin with the adherend fabric adhering thereto by reciprocating the roller on a metal plate once. After the pressure bonding, the initial adhesive strength of the sanitary napkin was measured. A tensile tester (TENSILON RTG-1225 produced by A&D Company, Limited) was used for the measurement. The adherend fabric was secured to an upper chuck of the tensile tester, and the sanitary napkin was secured to a lower chuck. In this state, T-peel testing was performed at a constant speed of 30 cm/min to measure the strength in peeling. At this time, there was no support by hand.

To measure adhesive strength after adhesive aging, the adherend fabric adhered to each of the sanitary napkins in the same manner as the above-described experiment. A pressure-bonding roller having a weight of 2 kg and a width of 65 mm was then used to pressure-bond the sanitary napkin with the adherend fabric adhering thereto by reciprocating the roller on a metal plate once. The sanitary napkin was then disposed between metal plates, and weights were placed on the metal plate in an evenly dispersed manner. In this state, the sanitary napkin was left at a temperature of 40° C. and a humidity of 80% for 24 hours. In this process, an average pressure of approximately 36 g/cm² was applied to the sanitary napkin. Thereafter, T-peel testing was performed with the tensile tester at room temperature in the same manner as the measurement of initial adhesive strength to measure the strength in peeling. At this time, there was no support by hand.

The adhesive strength after adhesive aging of the sanitary napkin for comparative examples was significantly greater than the adhesive strength immediately after adhering (adhesive strength immediately after adhering: approximately 0.2 N/57 mm, adhesive strength after adhesive aging: approximately 5 N/57 mm). The adhesive strength after adhesive aging of the sanitary napkin for working examples was also greater than the adhesive strength immediately after adhering; however, the degree of the increase in the adhesive strength was small (adhesive strength immediately after adhering: approximately 0.2 N/57 mm, adhesive strength after adhesive aging: approximately 2 N/57 mm). It is assumed that if the commercial sanitary napkin remains in an adhering state, the adhesive strength significantly increases, causing a residual adhesive or the like. In contrast, it is assumed that a residual adhesive can be reduced to a relatively small degree in the sanitary napkin including the pressure-sensitive adhesive fastener because the protruding members prevent a shift in position and accordingly such a strong adhesive strength is not required and because the protruding members prevent an increase in the adhesive strength if the sanitary napkin remains in an adhering state.

### Experiment for Evaluation of Residual Pressure-Sensitive Adhesive

This experiment is for measurement of a residual pressure-sensitive adhesive of a sanitary napkin on an adherend fabric. In this experiment, the same type of the commercial sanitary napkin as in the above-described experiment was prepared as a comparative example. A sanitary napkin including the same pressure-sensitive adhesive fasteners as in the above-described experiment was prepared as a working example. A cut piece of commercial underwear (sanitary panties) was prepared as an adherend fabric.

To evaluate the residual pressure-sensitive adhesive, the sanitary napkin and the adherend fabric adhered to each other. The sanitary napkin and the adherend fabric were then disposed between two metal plates and left at a temperature of 40° C. and a humidity of 80% for 24 hours while an average pressure of approximately 36 g/cm² was applied thereto. Thereafter, the sanitary napkin was peeled from the adherend fabric at a temperature of 40° C. and a humidity of 80% and at a speed of approximately 50 cm/min, and the presence or absence of a residual pressure-sensitive adhesive on the adherend fabric was evaluated visually and by touching with a finger.

The results of the experiment confirmed a residual pressure-sensitive adhesive on the adherend fabric with the sanitary napkin for comparative examples adhering thereto. In contrast, no residual pressure-sensitive adhesive was confirmed on the adherend fabric with the sanitary napkin for working examples adhering thereto.

Examples of preferred aspects for carrying out the present invention have been described; however, dimensions, materials, ways of adhesion, applications, and the like can be modified within the spirit of the present invention.

In a distribution channel, a so-called upstream company may distribute a component (also referred to as a fastener) including the protruding members 130 and the sheet base material 110 but not including a pressure-sensitive adhesive, and a so-called downstream company that is close to the user side and produces sanitary napkins and the like may add the pressure-sensitive adhesive layers 121, 122 and distribute an end product, such as a sanitary napkin. The pressure-sensitive adhesive layer 121 is preferably applied between the protruding members 130 from the viewpoint of space saving; however, the region of the pressure-sensitive adhesive layer 121 and the region of the protruding members 130 may be arranged side by side (This arrangement includes an adjacent arrangement).

### REFERENCE SIGNS LIST

1 Sanitary napkin
100 Pressure-sensitive adhesive fastener
110 Sheet base material
120, 121, 122 Pressure-sensitive adhesive layer
130, 130A, 130B, 130C, 130D, 130E, 130F Protruding member
131 Column section
131c Tip surface
132 Insertion section
132a Edge
132b First lateral side
132c Second lateral side
140 Boundary
146 Wide portion
147 Narrow portion

1. A pressure-sensitive adhesive fastener being overlapped with a target object to be coupled with the target object, the pressure-sensitive adhesive fastener comprising:
a sheet base material including a pair of facing surfaces;
a pressure-sensitive adhesive layer being formed on at least one of the surfaces of the sheet base material; and
a plurality of protruding members protruding from the at least one surface of the sheet base material in a first direction and being arranged so as to be separated from each other in a second direction being an expanding direction of the one surface;
the protruding members each comprising:
a column section extending from the sheet base material in the first direction and protruding from a front surface of the pressure-sensitive adhesive layer;
an insertion section protruding from a tip portion of the column section in the second direction and being inserted into the target object;
wherein the insertion section comprises an edge on an outer peripheral side in the second direction, a first lateral side being disposed on a distal end side of the column section with respect to the edge, and a second lateral side being disposed on a proximal end side of the column section with respect to the edge;
the edge is composed of a corner between the first lateral side and the second lateral side; and
the corner is at an acute angle in a cross section cut in the first direction.

2. The pressure-sensitive adhesive fastener according to claim 1,
wherein the insertion section comprises an edge on an outer peripheral side in the second direction, a first lateral side being disposed on a distal end side of the column section with respect to the edge, and a second lateral side being disposed on a proximal end side of the column section with respect to the edge; and
a tip surface of the column section and the first lateral side of the insertion section form a continuous surface together.

3. The pressure-sensitive adhesive fastener according to claim 2, wherein the tip surface of the column section and the first lateral side of the insertion section form a same plane.

4. (canceled)

5. The pressure-sensitive adhesive fastener according to claim 1, wherein the second lateral side is formed into an inward recess in the cross section cut in the first direction.

6. The pressure-sensitive adhesive fastener according to claim 1,
wherein the insertion section comprises an edge on an outer peripheral side in the second direction, a first lateral side being disposed on a distal end side of the column section with respect to the edge, and a second lateral side being disposed on a proximal end side of the column section with respect to the edge; and
at least one of the first lateral side and the second lateral side is formed into an outward protrusion in a cross section cut in the first direction.

7. The pressure-sensitive adhesive fastener according to claim 1, wherein the pressure-sensitive adhesive fastener has a dynamic shear strength of 7 N/6.45 cm² or greater on a cotton cloth.

8. A pressure-sensitive adhesive fastener being overlapped with a target object to be coupled with the target object, the pressure-sensitive adhesive fastener comprising:
a sheet base material including a pair of facing surfaces;
a pressure-sensitive adhesive layer being formed on at least one of the surfaces of the sheet base material; and
a plurality of protruding members protruding from the at least one surface of the sheet base material in a first direction and being arranged so as to be separated from each other in a second direction being an expanding direction of the one surface;
the protruding members each comprising:
a wide portion being widened in the second direction on a distal end side; and
a narrow portion being narrower than the wide portion in the second direction on a proximal end side;
the wide portion having a width of 250 μm or less;
the narrow portion having a width of 95% or less of the width of the wide portion; and
a difference between an average height of the protruding members and an average thickness of the pressure-sensitive adhesive being 250 μm or less,
wherein the wide portion comprises an edge on an outer peripheral side in the second direction, a first lateral side being disposed on a distal end side of the column section with respect to the edge, and a second lateral side being disposed on a proximal end side of the column section with respect to the edge;
the edge composed of a corner between the first lateral side and the second lateral side; and
the corner is at an acute angle in a cross section cut in the first direction.

9. An absorbent article comprising the pressure-sensitive adhesive fastener according to claim 8.

10. An adhesive material adhering to clothing, comprising the pressure-sensitive adhesive fastener according to claim 8.

11. An adhesive material adhering to fabric, comprising the pressure-sensitive adhesive fastener according to claim 8.

12. A fastener being overlapped with a target object to be coupled with the target object, the fastener comprising:
a sheet base material including a pair of facing surfaces; and
a plurality of protruding members protruding from at least one of the surfaces of the sheet base material in a first direction and being arranged so as to be separated from each other in a second direction being an expanding direction of the one surface;
the protruding members each comprising:
a column section extending from the sheet base material in the first direction; and
an insertion section protruding from a tip portion of the column section in the second direction and being inserted into the target object.

13. (canceled)

14. An absorbent article comprising the fastener according to claim 12.

15. An adhesive material adhering to clothing, comprising the fastener according to claim 12.

16. An adhesive material adhering to fabric, comprising the fastener according to claim 12.
